(19) United States
(12) Patent Application Publication
(10) Pub. No.: US 2021/0212952 A1
(43) Pub. Date: Jul. 15, 2021

(54) METHOD AND ENCAPSULATED SUBSTANCE FOR TREATING PAIN DUE TO A VARIETY OF DISEASES

(71) Applicant: Cuong Trinh, San Jose, CA (US)

(72) Inventor: Cuong Trinh, San Jose, CA (US)

(21) Appl. No.: 17/143,330

(22) Filed: Jan. 7, 2021

Related U.S. Application Data
(60) Provisional application No. 62/961,320, filed on Jan. 15, 2020.

(51) Int. Cl. A61K 9/48; A61K 9/00; A61K 31/485

(52) U.S. Cl. CPC A61K 9/4808; A61K 31/485; A61K 9/0053

(57) ABSTRACT

The invention is a method for pain relief due to rheumatoid arthritis, dermatitis, Crone's disease, fibromyalgia and multiple sclerosis. It comprises a single dose of LDN-α, a biphasic formulation of naltrexone, C₂₀H₂₃NO₄, taken daily. Proportions of LDN-α components may be varied over a range so as to achieve a desired level of naltrexone in the blood for a period of 19 to 27 hours.

### TECHNICAL FIELD

The invention is a method involving using an encapsulated substance for treating pain due to rheumatoid arthritis, dermatitis, Crone's disease, fibromyalgia and multiple sclerosis.

### BACKGROUND OF THE INVENTION

Rheumatoid arthritis is an auto-immune disease whose cause is unknown and which is both painful and debilitating. Dermatitis involves inflammation of the skin resulting in redness, itchiness and rash. Crone's disease is an inflammatory bowel disease that can cause abdominal pain. Fibromyalgia is painful. Its cause is unknown but symptoms include chronic pain throughout the body and physical-pressure sensitivity. Multiple sclerosis is a disease of the central nervous system. The immune system attacks myelin, the protective sheath around nerve fibers. As with fibromyalgia, treatment for pain relief due to multiple sclerosis varies with varied success of each.

Naltrexone is a medication approved by the FDA and prescribed as a treatment for addiction due to opioids and alcohol. It is commonly provided in tablets of 50 or 100 mg. It has been found that low-dosage naltrexone (LDN) may relieve pain due to rheumatoid arthritis, dermatitis, Crone's disease, fibromyalgia and multiple sclerosis, however, it has not yet been approved for that purpose by the FDA and is unavailable in tablets or capsules in low-dosage quantities. As such, when it has been prescribed for such pain relief treatment, it is obtained from compounding pharmacies that create capsules having lower amounts of naltrexone.

Naltrexone provided in low-dosage compounded form has been immediate-release type and, when taken orally, is absorbed (96 percent) into the blood stream within one hour and peak levels are found within one hour of dosing. It is also metabolized in the liver within a half-life of six hours. Thus, as presently provided LDN compounded medications, unless taken multiple times per day, the levels of naltrexone in the blood may vary significantly which may affect the degree of pain relief, as well.

### BRIEF DESCRIPTION OF THE INVENTION

The invention herein described and claimed is a method for reducing pain due to rheumatoid arthritis, dermatitis, Crone's disease, fibromyalgia or multiple sclerosis with dosage to once per day while keeping levels of naltrexone in the blood varying over a much smaller degree. When compounding a capsule for treatment of pain in fibromyalgia and multiple sclerosis, by using a combination of immediate-release and controlled-release components, one can tailor the dosage such that a desired level of naltrexone is achieved within the first hour, and essentially maintained at an effective level during the dosage duration.

As such a formulation of immediate-release naltrexone of X mg combined with controlled-release naltrexone of Y mg can maintain a level of naltrexone in the blood with desired limits during a dosing period. For example, given the half-life of naltrexone and the rate of controlled release, significant peaks and valleys during the dosage period may be avoided while some minimum level is maintained and a maximum level is not exceeded. In that case, the pain-relief benefit of the LDN capsule so formulated remains essentially steady after initial uptake.

In this method, the single dose of compounded LDN with a combination of immediate-release and controlled-release components is meant to be taken at the same time, once per day. The total amount of naltrexone is 4.5 mg. The immediate-release component may vary from 4.0 to 0.5 mg, and that of the controlled-release component may vary from 0.5 to 4.0 mg. In any case, the combination would be 4.5 mg. The relative proportions can be determined by the desired level of naltrexone in the blood over during a dosage period.

A capsule compounded such that 3.5 mg is immediate release and 1 mg is controlled release, given the half-life of naltrexone, would provide sufficient naltrexone blood levels for pain relief for as long as 27 hours between dosages. It would also support taking a dose 19 hours or more after a previous dose without risking exceeding a maximum level of naltrexone in the blood.

### BRIEF DESCRIPTION OF DRAWINGS

FIG. 1 shows a two-dimensional view of the structure and constitution of naltrexone.

FIG. 2 shows the rate of absorption and effect of half life on a 50 mg oral dose of naltrexone.

FIG. 3 shows when controlled-release components are absorbed into the blood stream it has the effect of keeping the level of naltrexone in the blood at a higher level than would be the case with immediate-release components only.

### DETAILED DESCRIPTION OF INVENTION

Rheumatoid arthritis, dermatitis, Crone's disease, fibromyalgia and multiple sclerosis are chronic diseases that can cause significant, persistent pain. There are no cures for these diseases and physicians prescribe a variety of medications for pain relief for each of them, and successful pain relief has been spotty.

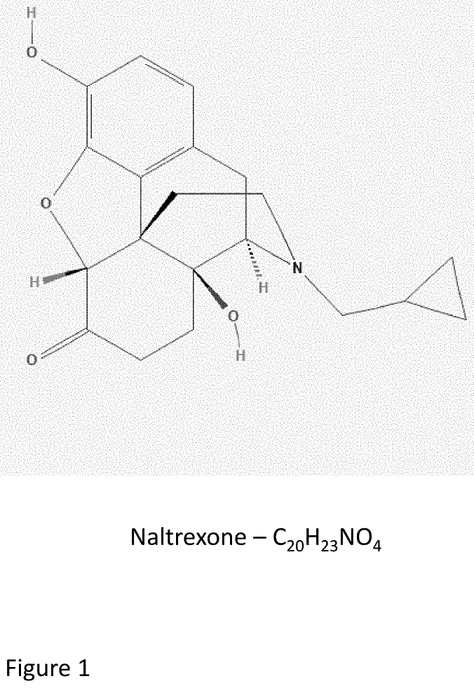

A medication called “naltrexone” (FIG. 1) is approved by the FDA for treatment of addiction to both alcohol and opioids. Its chemical formula is C₂₀H₂₃NO₄. Sources of the drug provide tablets and capsules of typically 50 to 100 mg. However, recent research has shown that low-dose naltrexone (LDN) has provided pain relief for sufferers of fibromyalgia and multiple sclerosis.

However, because available medication is typically 50 to 100 mg, LDN medications today are made by compounding pharmacists. And, to date, those medications have been made using immediate-release LDN components.

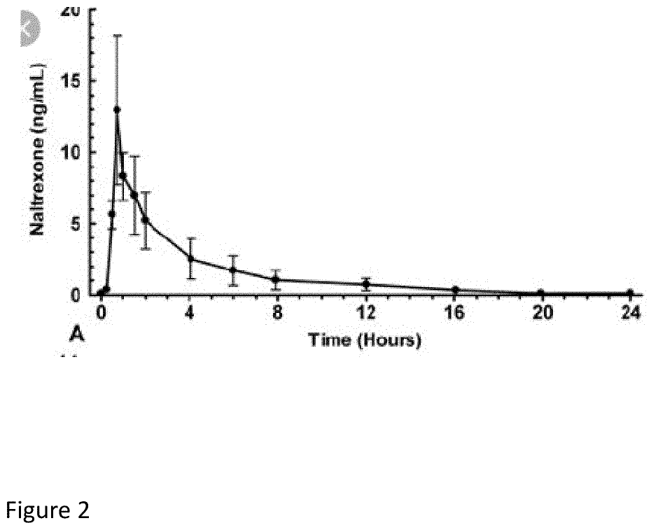

One result of using immediate-release LDN for treating fibromyalgia and multiple sclerosis pain is significant peaks and nulls in naltrexone levels in the blood stream during each 24-hour period (FIG. 2).

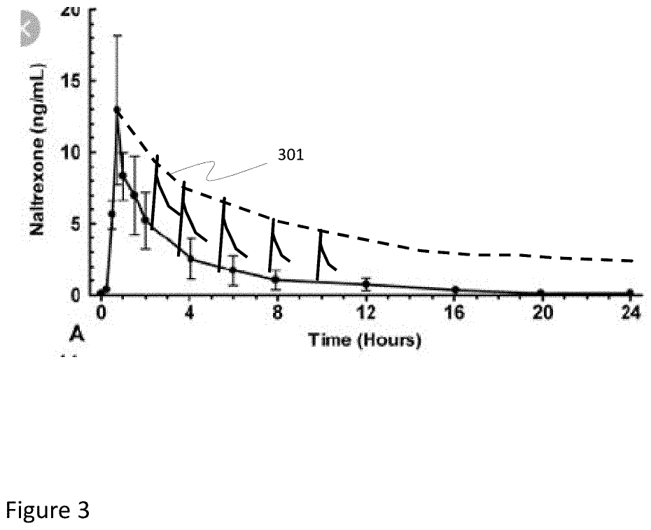

The invention herein disclosed and claimed is a method for relieving pain due to fibromyalgia and multiple sclerosis wherein dosage is a capsule comprising both immediate-release and controlled-release components of low-dosage naltrexone, or biphasic delivery. In FIG. 3, the controlled-release components periodically bolster the level of naltrexone in the blood (curve 301) such that at the end of 24 hours the difference in level with the biphasic substance varies from 12 to 5 versus 20 to less than 2. By controlled-release, these components may comprise the various means and delivery rates associated with controlled-release. To distinguish between LDN and this new formulation, it will be referred to as LDN-α to indicate a biphasic encapsulated formulation.

The method comprises ingesting a capsule containing the biphasic compounded naltrexone, LDN-α, after which digestive action on the capsule's container exposes the biphasic naltrexone to absorption into the blood stream. The components of the capsule that comprise immediate-release components will be absorbed, first, and within the first hour, cause an initial peak in naltrexone levels in the blood stream. Over a day-long period, the controlled-release components are exposed to absorption at later times. Thus, rather than naltrexone variation as shown in FIG. 2, the slower delivery of controlled-release components keep the naltrexone level in the blood stream at essentially a rate wherein variances are confined to much smaller changes (FIG. 3, 301).

The novelty of the biphasic formulation of naltrexone as applied to a method of pain relief is its combination of immediate-release and controlled-release performance. Furthermore, the proportions of immediate-release and controlled-release components may be varied more specifically to an individual patient to adjust the naltrexone level in the blood during each daily dosage period. By selecting a conservative maximum and minimum level of naltrexone in the blood, one is able to adjust the proportions of components to achieve an effective yet safe level. Because the medication is made by compounding, it is more practical to enable this degree of proportion tailoring wherein a mass-produced biphasic product would be more practical where a particular proportion is found that suits a large group of users.

The novelty of the LDN-α is by virtue of its biphasic characteristics and the ability to adapt different proportions of immediate-release and controlled-release components due to the smaller scale, manual processes involved in making the product.

The preferred formulation is one wherein the immediate-release component in each capsule is between 4.0 and 0.5 mg, and where the controlled-release component is between 0.5 and 4.0 mg. In all cases, the total amount of all naltrexone in the capsule is 4.5 mg.

Tests show that a formulation having 3.5 mg of immediate-release components and 1.0 mg of controlled-release components would provide an essentially consistent naltrexone level in the blood beginning approximately one hour after ingesting the capsule and for up to 27 hours. If taken at approximately the same time, daily, the capsule would provide consistent naltrexone levels and commensurate pain relief during that duration.

What is claimed is:

1. A pain-relief method for pain due to rheumatoid arthritis, dermatitis, Crone's disease, fibromyalgia, or multiple sclerosis comprising:
a. ingesting LDN-α capsule at a time, T;
b. dissolving the capsule containment by action of gastric juices thereby releasing components of LDN-α into stomach;
c. absorbing immediate-release components of LDN-α into blood stream;
d. absorbing, gradually, controlled-release components of LDN-α into blood stream; and
e. repeating a through d at time, T+24 hours later on successive days.

2. An encapsulated formulation of naltrexone, LDN-α, comprising:
immediate-release-naltrexone components;
controlled-release-naltrexone components;
total weight of all naltrexone components is 4.5 mg;
weight of said immediate-release-naltrexone components is A;
weight of said controlled-release-naltrexone components is B; and
combination of said A and said B is 4.5 mg.

3. A claim as in 2 further comprising:
weight of said A may range between 4.0 and 0.5 mg;
weight of said B may range between 0.5 and 4.0 mg; and
total weight of A+B is 4.5 mg.
